(19) United States
(12) Patent Application Publication
(10) Pub. No.: US 2016/0282355 A1
(43) Pub. Date: Sep. 29, 2016

(54) MATERIALS AND METHODS FOR ANALYSING GLYCATION

(71) Applicant: THE UNIVERSITY OF BATH, Bath (GB)

(72) Inventors: Tony David James, Bath (GB); Stephen Edward Flower, Bristol (GB); Johannes Maria Hubertus van den Elsen, Bath (GB); Marta Patricia Pereira Morais, Stevenage (GB)

(21) Appl. No.: 14/778,297

(22) PCT Filed: Mar. 19, 2014
(86) PCT No.: PCT/GB2014/050862

§ 371 Date: Sep. 18, 2015

(30) Foreign Application Priority Data
Mar. 20, 2013 (GB) 1305114.9

(51) Int. Cl. G01N 33/58; G01N 33/68; G01N 27/447

(52) U.S. Cl. CPC G01N 33/582; G01N 27/44726; G01N 27/44739; G01N 27/44747; G01N 27/44782; G01N 33/6848; G01N 2440/38

(57) ABSTRACT

The present invention relates to materials, methods and kits for detecting and identifying glycated species, in particular methods in which glycated species are first labelled and then subjected to a separation step, following which the label may be directly detected.

### FIELD OF THE INVENTION

The present invention relates to materials, methods and kits for detecting and identifying glycated species, in particular for analysing glycation, and in particular using labelled boronic acid species for detecting and identifying glycated species.

### BACKGROUND OF THE INVENTION

Protein glycation, also known as non-enzymatic glycosylation, has been implicated in various disease states^{1,2} and is therefore an important biomarker for ageing and age-related chronic diseases such as diabetes, cardiovascular diseases, autoimmune diseases, cancer, and Alzheimer's disease (AD)^{3-8, 9-11}. This process whereby reducing sugar molecules, such as glucose, react with the amino groups of lysine, arginine or N-terminal amino acid residues of proteins ultimately leads to the formation of complex and stable advanced glycation endproducts (AGEs). This modification affects the folding, function and stability of long-lived proteins. The analysis of these non-enzymatic protein-carbohydrate adducts is challenging because of their complexity and variability.

A variety of analytical techniques are currently used to analyse the protein modifications resulting from glycation, each with their advantages and limitations. Boronate affinity chromatography (BAC)¹², for instance, is based on the interaction between boronic acid and cis-diol-containing carbohydrates. It can detect various types of glycated proteins and has been widely used, in low-performance agarose gel electrophoresis systems as well as high-performance chromatography-based systems¹³. It has been used for example to differentiate the HbA_{1c} isoform as a marker for diabetes and indicator for blood sugar control. With BAC all types of glycation modifications, as well as N- and O-linked glycosylation, in a sample are retained and the identification and analysis of individual glycated proteins requires further separation steps. BAC has been successfully used as a sample enrichment step prior to high-performance liquid chromatography (HPLC) and mass spectrometry (MS) analyses¹⁴. Even though MS¹⁵ is an ideal technique to determine the identity of protein band in a gel, the limited availability of database information specific for glycation adducts hampers the identification of the modifications. In addition, MS only detects the most abundant proteins and if only a small percentage of a given protein species in a sample has suffered glycation damage, these adducts are likely to go undetected. Furthermore, any information regarding the specific glycation state can be lost after digestion of the protein samples prior to analysis.

In order to provide a simple, cost-effective detection and analysis tool for glycated proteins, an electrophoresis based method using polyacrylamide incorporated with the carbohydrate affinity ligand methacrylamido phenylboronic acid (MPBA) was recently developed¹⁶, see also WO 2010/041037¹⁷. This technique was first tested on simple carbohydrates and showed that by using the same basic principle as BAC, exploiting the reversible covalent interaction between boronic acid and cis-diols^{18, 19}, the MPBA-incorporated acrylamide gels enabled the improved separation of saccharides, and the differentiation between monosaccharides and dissacharides. Later this boronate-assisted saccharide electrophoresis (BASE) method was adapted to allow the separation of glycated from non-glycated proteins by incorporating MPBA into polyacrylamide gels for sodium dodecyl sulphate polyacrylamide gel electrophoresis (SDS-PAGE) analysis^{20,21}. This method, coined mP-AGE, showed that under electrophoresis conditions the polyacrylamide incorporated MPBA is specific for fructosamine modified proteins, via interactions with the cis-1,2-diol-containing fructosamine adducts and by further stabilisation through an electrostatic interaction between the protonated amino group and the negatively charged boronate moiety²⁰. This method enables the differentiation between early and late glycated adducts and has now been successfully used to analyse glycated human serum albumin (HSA) in serum from diabetes sufferers²². Whilst mP-AGE aids the detection and separation of glycated proteins, it is only suitable for the analysis of samples with limited complexity.

EP 0455225 A describes methods for measuring the percentage glycation of a particular protein that include the use of a labelled phenyl boronic acid derivative. However, the method does not use direct detection of the labelled protein and separation of species occurs prior to a labelling step that is carried out by capture on a solid support with subsequent detection.

WO 2010/135574 describes methods for determining glycation of haemoglobin using boronic acids labelled with detectable markers in which an immobilised antibody against a specific glycated protein is used to capture the protein, followed by a subsequent step of labelling the bound protein with a labelled boronic acid species. Electrophoresis is only mentioned as a means to separate unbound antibody (first agent, not bound to haemoglobin) and the unbound boronic acid (second agent, not bound to glycated protein).

Accordingly, there remains an unmet need in the art for techniques that are amenable for the analysis of complex glycated samples and which overcome one or more of the drawbacks of the prior art techniques as described above.

### SUMMARY OF THE INVENTION

Broadly, the present invention concerns materials, methods and kits for detecting and identifying glycated proteins, and in particular glycated species, such as glycated proteins, in a variety of complex samples, using labelled boronic acid compounds to label the glycated species present in samples. In a some embodiments, the present invention uses boronic acid compounds to facilitate separation of glycated proteins from non-glycated proteins within a sample, and further to facilitate the separation of different glycated proteins. Without wishing to be bound by any particular theory, it is believed that interaction between polyhydric species and the boronic acid leads to reversible formation of boronate esters, labelling the polyhydric species. This interaction may occur during incubation of a sample with a suitable boronic acid compound and typically takes place prior to separation.

In preferred embodiments described below, the methods of the present invention employ fluorescent labelled boronic acid compounds in gel electrophoresis (Flu-PAGE) and Eastern blotting (Flu-BLOT) detection techniques.

Glycated proteins are important biomarkers for age-related disorders. However their analysis is challenging because of the complexity of the protein-carbohydrate adducts. Herein is described a method that enables the detection and identification of individual glycated proteins in complex samples using fluorescent boronic acids in gel electrophoresis. Using this method the present inventors have identified glycated proteins in human serum, insect hemolymph and mouse brain homogenates, confirming this technique as a powerful proteomics tool that can be used for the identification of potential disease biomarkers.

Accordingly, in a first aspect, the present invention provides a method of determining the presence of one or more glycated species in a sample, the method comprising labelling a sample suspected of containing the glycated species with a labelled boronic acid compound, wherein the labelled boronic acid compound is capable of specifically labelling glycated species present in the sample; separating glycated species present in the sample from non-glycated species; and detecting the label to determine the presence of the glycated species in the sample. Preferably, the labelling of the sample occurs through incubation of the sample with a suitable boronic acid compound, which may occur at room temperature. Less stable samples may be labelled overnight at lower temperatures as appropriate. The method may comprise identifying one or more glycated species in the sample and/or determining the amount or level of one or more glycated species in the sample. Preferably, the labelled boronic acid compound preferentially labels glycated species present in the sample and/or substantially does not label glycosylated and/or non-glycosylated species present in the sample. Without wishing to be bound to any particular theory, the present inventors believe that this indicates that the boronic acid species of the present invention specifically interact with fructosamine modified proteins. This offers advantage over other methods in which identification and analysis of individual glycated proteins requires further separation steps to separate the glycated proteins from N- and O-glycosylated species.

In some preferred embodiments, the step of separating the components of the sample comprises applying an electric field to the sample to cause different glycated components of the sample to migrate at different rates. For example, the step of separating the sample may be carried out by affinity electrophoresis, gel electrophoresis, capillary electrophoresis, dielectrophoresis, isotachophoresis, two-dimensional electrophoresis and/or mass spectrometry. Preferred methods include gel electrophoresis, capillary electrophoresis and mass spectrometry.

In some embodiments, detecting the glycated species may comprise transferring the glycated species to a membrane (blotting) and detecting the label. In other embodiments, the method may comprise detecting one or more species resolved or separated on a gel, for example, following separation using gel electrophoresis.

Detecting or identifying species present in the sample may be achieved using electrophoresis, chromatography, direct visualisation, protein staining, anti-body probing and/or mass spectrometry. In some embodiments, the mass spectrometry is MALDI-TOF mass spectrometry. One or more techniques may be used where appropriate. For example, direct visualisation may be followed by analysis of one of more bands using mass spectrometry and/or a further protein detection method such as protein staining/anti-body probing, for example a Western blot technique.

In some embodiments, the method comprises loading an electrophoresis gel with the labelled sample and applying an electric field across the gel to cause the glycated species to migrate across the gel. The presence of the boronic acid label may cause retardation of movement of labelled glycated species such as proteins, facilitating separation. The method may further comprise detecting one or more of the glycated species resolved or separated on the gel. In some preferred embodiments, the step of detecting the label comprises directly detecting the label.

Any label may be used to detect the polyhydric species resolved in methods according to the invention, and may be included in the kits of the invention. Neutral labels may be more desirable, because charged labels can affect the true nature of the polyhydric species. The label may be any label suitable to enable detection or visualisation of the polyhydric species resolved by the methods of the invention. Labels for use in the present invention may include labels suitable for detection in computed tomography (CT), single photon emission tomography (SPECT), positron emission topography (PET), magnetic resonance imagining (MRI), mass spectrometry (MS), and visual detection, for example using visible or UV light. Accordingly, examples of suitable labels include, but are not limited to, radiolabels, chromophores, phosphors including fluorophores, and chemiluminescent labels.

In some embodiments, the label is a visible or fluorescent label. In some preferred embodiments, the label is a fluorescent label. A particular advantage of the use of fluorescent labels is the potential for direct detection of the labels, and consequently of the labelled glycated samples. Fluorescence is the emission of light by a substance that has absorbed electromagnetic radiation. In preferred embodiments, the absorbed radiation is preferably ultraviolet light or visible light. Preferably the wave length of the visible light is in the blue region of the visible spectrum. Examples of fluorescent compounds are known in the art and include some dyes. Through the incorporation of such a fluorescent dye moiety into the labelled boronic acid compound in some embodiments of the present invention, the label may be directly detected by visualisation, for example, using a light box or other suitable apparatus. The direct visualisation may be image captured by a camera or similar. Suitable apparatus for UV or visible light visualisation and imaging are known in the art. In some embodiments, this visualisation occurs after transfer from, for example, the gel, to a membrane (blotting).

This Flu-BLOT method allows convenient and facile detection of bands associated with glycated proteins. The method is non-destructive, and allows for the further use of additional protein detection protocols. Suitable additional protocols and techniques may include analysis using mass spectrometry techniques, for example, Time of Flight techniques such as MALDI-TOF. A band of interest in the blot may be sampled and analysed, without the protein having been subjected to blocking, staining and/or antibody probing in order to be detected. Other suitable protein detection protocols include those known in the art, and may include, for example, Western blotting techniques.

Preferably, the step of labelling the sample substantially does not affect the electrophoretic migration properties of the glycated species.

Methods described herein may be capable of detecting glycated polypeptides in complex samples and preferably do not require the use of additional enrichment or purification techniques. The sample may comprise only a glycated component, multiple glycated components, or a mixture of glycated component(s) and corresponding non-glycated species. The glycated species may be a polypeptide and may, for example, comprise glycated glucose, mannose, fructose, maltose or galactose sugars. Preferably, the method may be used in the detection of early stage glycation adducts. Glycated species may glycated peptides, glycated polypeptides, glycated proteins, glycated nucleic acid such as glycated DNA or glycated RNA, glycated lipids and/or a mono-, oligo- and poly-saccharides.

As used herein, the term complex sample includes samples comprising more than five polypeptides in which at least one polypeptide is glycated. In some embodiments, the number of species in the sample is greater than five. In some embodiments, the number of species in the sample is greater than 10. In some embodiments, the number of species in the sample is greater than 20. In some embodiments, the number of species in the sample is greater than 50. In some embodiments, the number of species in the sample is greater than 100. One or more glycated species may be present in the complex samples of these embodiments. By way of example, and without limitation, in some embodiments the method may be used to detect a single glycated species in a complex sample comprising over 100 components such as proteins.

In some embodiments, the sample is from an animal model of protein glycation. Methods of the invention may also enable the study of glycated proteins in the context of other non-affected proteins in fluid (for example, human serum and insect hemolymph) and solid (for example, mouse brain cortex homogenates) biosamples. Accordingly, in some embodiments, the sample is a fluid sample. Examples of suitable fluid samples include, but are not limited to, blood, sera, saliva, cerebrospinal fluid (CSF), tear fluid, or hemolymph. In some embodiments, the sample is a solid sample, for example, a tissue homogenate. Examples of suitable tissue homogenate samples include, but are not limited to, those derived from skin, brain, heart, lungs, oesophagus, stomach, bladder, gallbladder, intestine, kidney, liver, pancreas, spleen, prostate, testicle, ovary, uterus, and prostate. In some embodiments, the tissue homogenate is derived from skin or brain tissue. In some embodiments, the tissue homogenate is derived from skin. In some embodiments, the tissue homogenate is derived from brain tissue.

In some embodiments, the sample is a food product. Analysis of the glycated content of food products is of interest in the consideration of the health benefits and risks associated with particular food groups, and may have utility in the correlation of the glycated content of certain food groups with the onset or exacerbation of certain age-related diseases and disorders. Accordingly, in some embodiments, the sample is a food sample and the glycated species are food advanced glycation end products. Accordingly, in some embodiments, the method further comprises the step of correlating the presence and/or amount of one or more glycated species in a food product to the risk of onset or exacerbation of certain diseases and disorders associated with consumption of that food product. Examples of suitable food products include, but are not limited to, cured meat products, cooked meat products (for example, barbecued, roasted, grilled or fried), vegetable products and other cooked food products (for example, baked goods).

In some embodiments, the sample is a product of recombinant protein production. Detection of glycation during recombinant protein production methods may have utility in, for example, methods for the production and analysis of protein therapeutics such as monoclonal antibodies.

Details of suitable boronic acid species for use according to the present invention are given below. Briefly, the labelled boronic acid compounds of the present invention comprise a label to enable detection of the polyhydric species resolved in methods according to the invention. Labels may include labels suitable for detection in computed tomography (CT), single photon emission tomography (SPECT), positron emission topography (PET), magnetic resonance imagining (MRI), mass spectrometry (MS), and visual detection, for example using visible or UV light. Accordingly, examples of suitable labels include, but are not limited to, radiolabels, chromophores, phosphors including fluorophores, and chemiluminescent labels. In some embodiments, the boronic acid moiety may be an alkyl or aryl boronic acid species bound to the label through a covalent bond or via a linker.

In some preferred embodiments of the present invention, the labelled boronic acid compound comprises a fluorescent label. This fluorescent label may include 2-AA (2-aminobenzoic acid), 2-AB (2-aminobenzamide), DMB (diamino-4,5-methyleneoxybenzene), ANTS (8-aminonaphthalene-1,3,6-trisulfonic acid), or ANSA (1-amino-4-naphthalene sulphonic acid); or may be a dye selected from a fluorone dye, a rhodamine dye, an acridine dye, a cyanine dye, an oxazin dye, a phenanthrine dye or a derivative thereof. Preferably, the fluorescent label is a fluorone dye or a rhodamine dye, for example, fluorescein or rhodamine B.

In some embodiments, more than one labelled boronic acid compound is used. Some labelled boronic acid compounds of different chemical structure interact differently with glycated species, and consequently may show varying affinities to different glycated species in the sample. Accordingly, in some embodiments, the sample suspected of containing the glycated species comprises more than one glycated species and the labelled boronic acid compounds used have different affinities for different glycated species, such that glycated species may be differentiated by their preferential association with a labelled boronic acid compound. This multiplexing method further facilitates the identification and/or differentiation of glycated species in the sample. Without wishing to be bound to any particular theory, the present inventors believe that the preferential affinity of certain boronic acid compounds for particular glycated species is governed by a combination of steric and electrostatic factors, and may in particular be attributed to π-π (pi to pi) stacking interactions, which may be face-to-face, face-to-edge or edge-to-face, between aryl groups of the labelled boronic acid compound, if present, and the glycated species.

A method according to the present invention may further comprise correlating the presence or amount of one or more of the glycated species as a marker of a disease, condition or biological process. In some embodiments, the disease, condition or biological process is selected from cancer, microbial infection, Alzheimer's disease, diabetes, cardiovascular disease and ageing, including diabetes-related ageing.

In a further aspect, the present invention provides a method for diagnosing a patient suspected of having a disease associated with a glycated species, the method comprising labelling a sample suspected of containing the glycated species with a labelled boronic acid compound, wherein the labelled boronic acid compound is capable of specifically labelling glycated species present in the sample; separating glycated components present in the sample from non-glycated components; detecting the label to determine the presence of the glycated species in the sample; and correlating the presence or amount of one or more of the polyhydric species as a marker of a disease or condition. In some embodiments, the disease or condition is cancer, a microbial infection, Alzheimer's disease, diabetes, cardiovascular disease and/or ageing, including diabetes-related ageing.

In a further aspect, the present invention provides a kit for determining the presence of one or more glycated species in a sample, the kit comprising a labelled boronic acid compound, wherein the labelled boronic acid compound is capable of specifically labelling glycated species present in the sample; instructions for labelling a sample suspected of containing the glycated species with the labelled boronic acid compound and detecting the label to determine the presence of the glycated species in the sample. In some embodiments, the instructions include the step of separating glycated components present in the sample from non-glycated components. In some embodiments, the kit further comprises a gel suitable for use in the present invention as described herein. Suitable gels are described herein and include any physical or chemical gel that can sieve and/or separate glycated and other species in an electric field.

Embodiments of the present invention will now be described by way of example and not limitation with reference to the accompanying figures. However various further aspects and embodiments of the present invention will be apparent to those skilled in the art in view of the present disclosure.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

### BRIEF DESCRIPTION OF THE FIGURES

FIG. 1. Flu-PAGE and Flu-Blot analysis of human serum. Normal human serum (lanes 1, 1′ and 1″) and serum samples labelled with fluorescein (lanes 2, 2′ and 2″), fluorescein-boronic acid (lanes 3, 3′ and 3″), rhodamine (lanes 4, 4′ and 4″) and rhodamine-boronic acid (lanes 5, 5′ and 5″) in a 12% non-denaturing polyacrylamide gel (A) and Eastern blot (B). The gels and blots were imaged with Dark Reader® (lanes 1-5), UV (365 nm, with orange filter (595 nm); lanes 1′-5′) and UV (365 nm, with green filter (537 nm); lanes 1″-5″). The Coomassie stained control lane is shown in the left panel, labelled as C. The asterisks indicate an extra band present in the rhodamine boronic acid labelled sample (lane 5′) on both the Flu-PAGE and Flu-Blot. (C) shows the Western blot analysis of glucose incubated HSA after 0 (lanes 1 and 2) and 28 days (lanes 3 and 4) using anti-AGE antibodies. Lanes 1 and 3 correspond to unlabelled samples, whereas lanes 2 and 4 are labelled with fluorescein-boronic acid.

FIG. 2. Flu-PAGE analysis of in vitro glycated serum. SDS-PAGE of human serum glycated with 50 mM sugar at 37° C. for 7 and 10 days, lanes 2-7 and 8-12 respectively. Shown are non-incubated control (lane 1) and samples incubated with glucose (lanes 2 and 8), fructose (lanes 3 and 9), mannose (lanes 4 and 10), maltose (lanes 5 and 11), galactose (lanes 6 and 12), sucrose (lane 7). The left panel shows Coomassie stained gel (samples 1-12) and the right panel shows the gel visualised with UV prior to staining (lanes 1′-12′). The arrows indicate fluorescent proteins identified by MS.

FIG. 3. Flu-PAGE analysis of Manduca sexta hemolymph. (A) Flu-PAGE analysis of fifth instar Manduca sexta hemolymph. Lanes 1-3 are unlabelled hemolymph and lanes 4-6 are samples labelled with fluorescein-boronic acid. The left panel shows the Coomassie stained samples (lanes 1-6) whilst the right panel shows gel visualised with Dark Reader® prior to protein staining (lanes 1′-6′). The arrows indicate fluorescent bands analysed by MALDI-TOF and proteins identified using Manduca sexta proteome database. Inset: comparison of Coomassie stained and Flu-PAGE stained section of the SDS-PAGE gel showing the differences in protein concentration and levels of glycation between three of the identified proteins. Indicated (white asterisk) is the position of Serpin 1, showing high levels of expression in hemolymph in the Coomassie stained gel and low levels of glycation with Flu-PAGE. (B) Flu-PAGE analysis of different developmental stages of Manduca sexta hemolymph labelled with fluorescein-boronic acid. Lanes 1-12 correspond to hemolymph extracted from larvae at 4, 6, 8, 10-18 days after hatching respectively. The left panel shows Coomassie stained gel (lanes 1-12) and the right panel shows visualisation using Dark Reader® prior to protein staining (lanes 1′-12′). Inset: glycation levels of some proteins vary dramatically during development. Phenoloxidase subunit 1A (indicated with white asterisk), a highly expressed protein in Manduca sexta hemolymph, shows low levels of glycation on day 16 and 17 and high levels of glycation on day 18, on advancement to pupa stage.

FIG. 4. Flu-PAGE analysis of TASTPM mouse cortex homogenates. Flu-PAGE gel profile of TASTPM cortex homogenates, visualised with Dark Reader® (right panel, lanes 2′ and 3′) prior to Coomassie staining (left panel, lanes 1-3). The solid arrows indicate the positions of the proteins that have been identified using MALDI-TOF analysis. The dotted arrows represent higher molecular weight fluorescent proteins to be investigated.

FIG. 5. Chemical structures of the fluorescent labels. (A) Fluorescein (left) and fluorescein-boronic acid (right), (B) rhodamine (left) and rhodamine-boronic acid (right) structures generated using ChemDraw (CambridgeSoft).

FIG. 6. Fluorescein-boronic acid labelled serum analysed by mP-AGE. mP-AGE gel profile of human serum labelled with fluorescein-boronic acid when imaged with UV prior to protein staining (right) and after Coomassie stain (left).

FIG. 7. Flu-PAGE analysis of diabetic serum. (A) 8% SDS-PAGE of fluorescein (lanes 2-5) and fluorescein boronic acid labelled samples (lanes 7-11), control serum (lanes 2 and 7) and type 1 diabetes human sera of three individuals (lanes 3-5 and 8-10). Lanes 1 and 6 show the labels of fluorescein and fluorescein-boronic acid respectively. Lane 11 shows unlabelled control human serum. Gel was visualised and imaged with UV (365 nm and green filter 537 nm) on Alphalmager (right), and Coomassie stained (left). (B) shows relative fluorescence intensity of the HSA band in serum samples labelled with fluorescein-boronic acid. The fluorescence intensity values are corrected for protein concentration as determined by Coomassie stain, using TotalLab Quant.

FIG. 8. Quantitative fluorescence intensity analysis of Manduca sexta hemolymph. Comparative analysis of protein band intensities of hemolymph SDS-PAGE profiles in Flu-PAGE (A) and subsequent Coomassie stained gel (B). The gel intensity profiles shown were produced using TotalLab Quant. The identified intensity peaks correspond to 1) apolipophorin precursor protein, 2) pro-phenoloxidase subunit 2, 3) pro-phenoloxidase subunit 1, 4) hemocyte aggregation inhibitor protein precursor, 5) serpin 1, 6) putative C1A cysteine protease precursor and 7) insecticyanin. Graph C shows the fluorescence intensity of glycated protein bands (blue bars) that have been normalised with respect to their corresponding Coomassie stain intensity. The percentages of glycated lysines (see Table 1) in the respective proteins are shown as diamonds.

FIG. 9. Flu-PAGE analysis of TASTPM and wildtype mouse brain homogenates. Flu-PAGE analysis of wild type (A) and TASTPM (B) mouse cortex homogenates. The gel was visualised with Dark Reader® (left) prior to Coomassie staining (right). There are many more fluorescent protein bands in the TASTPM affected mouse sample compared to the age-matched control. Proteins that were analysed by MS were indicated.

### DETAILED DESCRIPTION

Boronic Acid Species

The synthesis of labelled boronic acid compounds suitable for use in accordance with the present invention is disclosed herein and other examples are available to the skilled person from the prior art and include boronic acids that are capable of forming cyclic boronic esters with various glycated species under equilibrium conditions, via reversible covalent interactions in aqueous media. References to boronic acids include anhydrides thereof, in particular the corresponding cyclic trimeric anhydride. Anhydrides may form during synthesis or during storage and may be present in any quantity, from trace to substantial amounts. These anhydrides typically exhibit substantially identical reactivity to the corresponding boronic acid.

The range of boronic acid compounds that can be employed in the present invention includes C₁₋₂₀-alkyl boronic acids and aryl boronic acids, which may be substituted or unsubstituted. In some embodiments, the boronic acid compounds are selected from C₁₋₂₀-alkyl boronic acids and substituted or unsubstituted aryl boronic acids. In some embodiments, the boronic acid compounds are selected from C₁₋₆-alkyl boronic acids and substituted or unsubstituted aryl boronic acids.

In some embodiments, the boronic acid compounds are substituted or unsubstituted aryl boronic acids, wherein aryl can be carboaryl or heteroaryl. In some embodiments, the boronic acid compounds are phenyl boronic acids.

The boronic acid compounds comprise a label. The label may be any label suitable for detection in computed tomography (CT), single photon emission tomography (SPECT), positron emission topography (PET), magnetic resonance imagining (MRI), mass spectrometry (MS), and visual detection, for example using visible or UV light. Accordingly, examples of suitable labels include, but are not limited to, radiolabels, chromophores, phosphors including fluorophores, and chemiluminescent labels. In some embodiments, the boronic acid moiety is an alkyl or aryl boronic acid species bound to the label through a covalent bond or via a linker.

In some embodiments, the boronic acid compound is substituted with a fluorescent label (comprising a bound fluorophore). Suitable fluorophores include rhodamine and fluorescein and derivatives thereof, for example, rhodamine B, rhodamine 101, rhodamine 110, rhodamine 123, rhodamine-6G, and 2,4,5,7-tetraiodofluorescein. Other suitable fluorophores may include derivatives of acridine dyes, cyanine dyes, other fluorone dyes, for example, erythrosine, eosin, merbromin; oxazin dyes, and phenanthridine dyes. Other suitable fluorophores are available to the skilled person from the prior art and may include 2-AA (2-aminobenzoic acid), 2-AB (2-aminobenzamide), DMB (diamino-4,5-methyleneoxybenzene), ANTS (8-aminonaphthalene-1,3,6-trisulfonic acid), or ANSA (1-amino-4-naphthalene sulphonic acid).

The fluorescent label may be bound directly to the aryl boronic acid (by a covalent bond), or it may be bound by a linker. Suitable linkers include, but are not limited to, esters, amides, thioamides, carbonates, carbamates, thiocarbamates, ureas, and thioureas. In some embodiments, the linker is a thiocarbamate or a thiourea, preferably a thiourea. In embodiments in which the boronic acid is a phenyl boronic acid, the fluorescent label substituent may be ortho, meta, or para relative to the —B(OH)₂ group.

Accordingly, in some embodiments, the labelled boronic acid compound is a compound of formula (I):

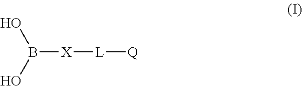

wherein:
X is C₁₋₂₀-alkylene optionally substituted with one or more R¹,
   C₆₋₁₀-carboarylene optionally substituted with one or more R²,
or
   C₅₋₁₀-heteroarylene optionally substituted with one or more R²;

L is:
a bond,
—(CO)O—, —O(CO)—, —NH(CO)—, ——(CO)NH—, —NH(CS)—, —(CS)NH—, —O(CO)O—, —O(CO)NH—, —NH(CO)O—, —O(CS)NH—, —NH(CS)O—, —NH(CO)NH—, or —NH(CS)NH—; and

Q is a label, preferably a fluorescent label;

wherein
each R¹, if present, is independently F, Cl, Br, I, CF₃, OH, OR^{A}, OCF₃, C₆₋₁₀-carboaryl or C₅₋₁₀-heteroaryl, wherein each C₆₋₁₀-carboaryl or C₅₋₁₀-heteroaryl, if present, is optionally substituted;
each R², if present, is independently F, Cl, Br, I, R^{A}, CF₃, OH, OR^{A}, OCF₃, C₆₋₁₀-carboaryl or C₅₋₁₀-heteroaryl, wherein each C₆₋₁₀-carboaryl or C₅₋₁₀-heteroaryl, if present, is optionally substituted;
each R^{A}, if present, is C₁₋₆-alkyl.

C₁₋₂₀ alkylene: The term “C₁₋₂₀ alkylene”, as used herein, pertains to a bivalent moiety obtained by removing two hydrogen atoms from a C₁₋₂₀hydrocarbon compound having from 1 to 20 carbon atoms, which may be aliphatic or alicyclic, linear or branched, or a combination thereof, and which may be saturated, partially unsaturated, or fully unsaturated.

C₁₋₆alkyl: The term “C₁₋₆alkyl”, as used herein, pertains to a monovalent moiety obtained by removing a hydrogen atom from a C₁₋₂₀ hydrocarbon compound having from 1 to 20 carbon atoms, which may be aliphatic or alicyclic, or a combination thereof, and which may be saturated, partially unsaturated, or fully unsaturated.

Examples of saturated linear C₁₋₆alkyl groups include, but are not limited to, methyl, ethyl, n-propyl, n-butyl, n-pentyl, and n-hexyl.

Examples of saturated branched C₁₋₆alkyl groups include, but are not limited to, iso-propyl, iso-butyl, sec-butyl, tert-butyl, and neo-pentyl.

Examples of saturated alicyclic C₁₋₆alkyl groups (also referred to as “C₃₋₆cycloalkyl” groups) include, but are not limited to, groups such as cyclopropyl, cyclobutyl, cyclopentyl, and cyclohexyl, as well as substituted groups (e.g., groups which comprise such groups), such as methylcyclopropyl, dimethylcyclopropyl, methylcyclobutyl, dimethylcyclobutyl, methylcyclopentyl, and cyclopropylmethyl.

Examples of unsaturated C₁₋₆alkyl groups which have one or more carbon-carbon double bonds (also referred to as “C₂₋₆alkenyl” groups) include, but are not limited to, ethenyl (vinyl, —CH═CH₂), 2-propenyl (allyl, —CH—CH═CH₇), isopropenyl (—C(CH₃)═CH₂), butenyl, pentenyl, and hexenyl.

Examples of unsaturated C₁₋₆alkyl groups which have one or more carbon-carbon triple bonds (also referred to as “C₂₋₆alkynyl” groups) include, but are not limited to, ethynyl (ethinyl) and 2-propynyl (propargyl).

Examples of unsaturated alicyclic (carbocyclic) C₁₋₆ alkyl groups which have one or more carbon-carbon double bonds (also referred to as “C₃₋₆cycloalkenyl” groups) include, but are not limited to, unsubstituted groups such as cyclopropenyl, cyclobutenyl, cyclopentenyl, and cyclohexenyl, as well as substituted groups (e.g., groups which comprise such groups) such as cyclopropenylmethyl.

C₆₋₁₀-carboarylene pertains to a bivalent radical obtained by removing a hydrogen atom from two ring atoms of a C₆-₁₀ carboaromatic compound, said compound having one ring or two rings. Examples of C₆₋₁₀-carboarylene include, but are not limited to, bivalent moieties derived from benzene (—C₆H₄—, phenylene) or naphthalene (—C₁₀H₆—, naphthylene).

C₅₋₁₀-heteroarylene pertains to a bivalent moiety obtained by removing a hydrogen atom from two ring atoms of a C₅₋₁₀ heteroaromatic compound, said compound having one ring or two rings and having from 5 to 10 ring atoms, of which from 1 to 5 are ring heteroatoms. Ring heteroatoms may preferably be selected from the group consisting of O, N, S and P. “C₃-₁₀” denotes ring atoms, whether carbon atoms or heteroatoms.

Examples of C₅₋₁₀-heteroarylene groups include, but are not limited to, C₅ heteroarylene moieties derived from furan, thiophene, pyrrole, imidazole, pyrazole, triazole, oxazole, isoxazole, thiazole, isothiazole, oxadiazole, and oxatriazole; and C₆ heteroaryl moieties derived from isoxazine, pyridine, pyridazine, pyrimidine, pyrazine, triazine, tetrazole, and oxadiazole.

In some embodiments, the labelled boronic acid compound is a compound of general formula (I):

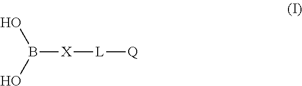

wherein:

X is C₆₋₁₀-carboarylene or C₅₋₁₀-heteroarylene and is optionally substituted with one or more R² groups;

each R² is independently F, Cl, Br, I, R^{A}, CF₃, OH, OR^{A}, OCF₃, wherein R^{A} is C₁₋₆-alkyl;

L is:
a bond,
—(CO)O—, —O(CO)—, —NH(CO)—, —(CO)NH—, —NH(CS)—, —(CS)NH—, —O(CO)O—, —O(CO)NH—, —NH(CO)O—, —O(CS)NH—, —NH(CS)—, —NH(CO)NH—, or —NH(CS)NH—; and

Q is a fluorescent label.

In some embodiments, the labelled boronic acid compound is a compound of formula (I) wherein X is phenylene optionally substituted with one or more R² substituents.

In some embodiments, the labelled boronic acid compound is a compound of formula (I) wherein X is selected from Ar^{P}, Ar^{M}, or Ar^{O}:

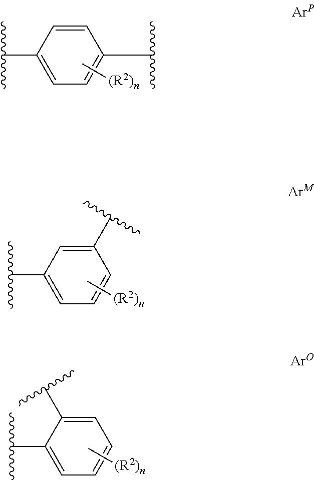

wherein n is a number between 0 and 4.

In some embodiments, n is 0, that is, no R² substituent is present.

In some embodiments, the labelled boronic acid compound is a compound of formula (I) wherein X is Ar^{M}. In some embodiments, the labelled boronic acid compound is a compound of formula (I) wherein X is Ar^{M} and n is 0.

In some embodiments, the labelled boronic acid compound is a compound of formula (I) wherein X is Ar^{M}, n is 0 and L is selected from —O(CS)NH— or —NH(CS)NH—.

In some preferred embodiments, the labelled boronic acid compound is a compound of formula (I) wherein X is Ar^{M}, n is 0, and L is —NH(CS)NH—.

In some preferred embodiments Q is a fluorone dye or a rhodamine dye.

In a preferred embodiment, the labelled boronic acid compound is Compound-A:

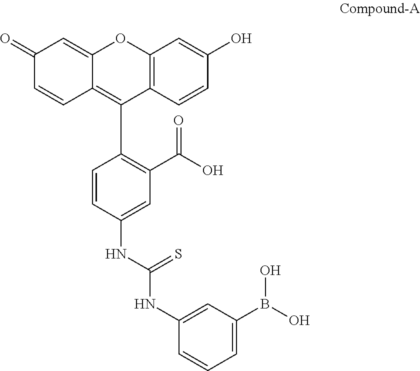 [SMILES: O=C(O)c1cc(NC(=S)Nc2cccc(B(O)O)c2)ccc1-c1c2ccc(=O)cc-2oc2cc(O)ccc12]

In a preferred embodiment, the labelled boronic acid compound is Compound-B:

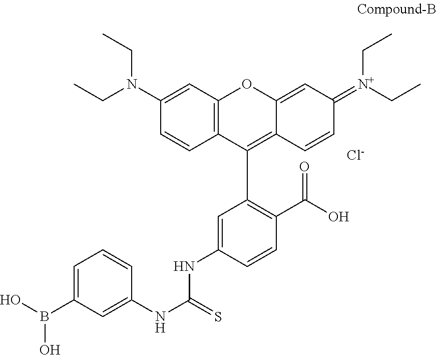 [SMILES: CCN(CC)c1ccc2c(-c3cc(NC(=S)Nc4cccc(B(O)O)c4)ccc3C(=O)O)c3ccc(=[N+](CC)CC)cc-3oc2c1 ; [Cl-]]

In a preferred embodiment, the labelled boronic acid compound is Compound-C:

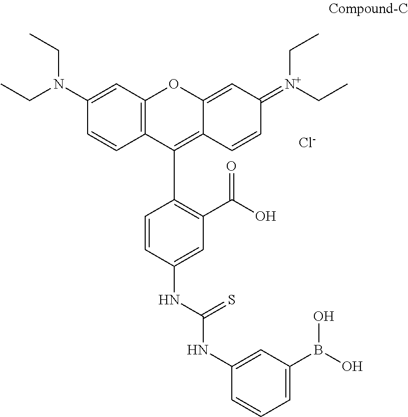 [SMILES: CCN(CC)c1ccc2c(-c3ccc(NC(=S)Nc4cccc(B(O)O)c4)cc3C(=O)O)c3ccc(=[N+](CC)CC)cc-3oc2c1 ; [Cl-]]

In a preferred embodiment, the labelled boronic acid compound is a mixture of Compound-B and Compound-C.

Suitable methods for the synthesis of these compounds will be readily apparent to one skilled in the art. For example, in a representative synthesis, an aryl boronic acid bearing a nucleophilic group such as an amino group, for example, 3-aminophenylboronic acid, undergoes an addition reaction with a fluorophore bearing a suitable substituent, for example, an isothiocyanate group.

Separation Techniques

As part of the methods of the present invention it may be desirable to separate glycated species present in a sample from non-glycated species, including glycosylated and/or non-glycosylated species. In general this can be achieved by separating the components of the sample by applying an electric field to the sample to cause different glycated components of the sample to migrate at different speeds. Suitable techniques for carrying this out include gel electrophoresis, capillary electrophoresis and/or mass spectrometry. In these methods, the method may comprise loading an electrophoresis gel with the labelled sample and applying an electric field across the gel to cause the glycated species to migrate across the gel. After separation, the method may comprise detecting the glycated species by transferring the glycated species to a membrane and detecting the label. Alternatively or additionally, the method may comprise detecting one or more of the glycated species resolved or separated on the gel.

The use of gel electrophoresis for separating biomolecules such as proteins and nucleic acids is well known in the art and the techniques disclosed in reference textbooks such as Maniatis and Sambrook (Molecular cloning : a laboratory manual, 3rd edition, New York: Cold Spring Harbor Laboratory, 2001) and Ausubel et al. (Short Protocols in Molecular Biology, 5th Edition, A Compendium of Methods from Current Protocols in Molecular Biology. Wiley, 2002) may be adapted for use in accordance with the present invention. In general, gel electrophoresis separates substances, most usually proteins, according to their electrophoretic mobility which is dependent on their size and length, molecular weight and other factors such as protein folding and post-translational modifications.

Gels suitable for use in the present invention include any physical or chemical gel that can sieve and separate glycated and other species in an electric field. Suitable gels include, but are not limited to, agarose gels and other derivatised celluloses, silica gel and acrylamide gels. A particularly preferred type of gel is those where the polymerisable linker is an acrylamide. Typically acrylamide linkers are used in combination with a polymerisable cross-linker such as a bisacrylamide monomer, for example methylene bisacrylamide, to produce electrophoresis gels. An initiator such as ammonium persulphate or TEMED is normally included to help to catalyse the polymerization reaction.

In use, gels may be used in reducing or non-reducing formats characterized by the inclusion (or not) of an agent such as sodium dodecyl sulphate (SDS) for denaturing proteins. These formats may also be used in the methods of the present invention. SDS is a long chain detergent that interacts with proteins and applies a negative charge that is in proportion to molecular weight, minimising the contribution made by the structure of proteins to their electrophoretic mobility so that migration is a function of molecular weight.

Glycated Species

As discussed above, the present invention relates to resolving glycated species. The glycated species which may be resolved by the methods of the present invention include those having a plurality of hydroxyl groups so that they can interact with the boronic acid species to reversibly form boronate esters or boronate ester analogues. Where the boronic acid species is a boronate ester, the polyhydric species may interact may interact with the boronic acid species by displacing the group forming the initial boronate ester. The boronate esters or boronate ester analogues formed by interaction of the polyhydric species with the boronic acid species may be cyclic. Boronate ester analogues include species wherein one or both of the O atoms of the boronate group are attached to an atom which is not C. By way of example, boronate ester analogues include boronate phosphoesters, which may be formed by the interaction between a boronic acid species, and one or more hydroxyl groups of a terminal phosphate.

It is preferable that the polyhydric species contains two hydroxyl groups which are sufficiently close to interact with a boronic acid species as discussed above. In particular, it may be desirable that the polyhydric species comprises two hydroxyl groups in a 1,1 or 1,2 or 1,3 or 1,4 positional relationship with each other. Hydroxyl groups in a 1,1 relationship are covalently attached to the same atom in the polyhydric species and those in a 1,2 relationship are covalently attached to adjacent atoms in the polyhydric species (i.e. atoms joined by one covalent bond). Similarly, hydroxyl groups in a 1,3 relationship are attached to atoms in the polyhydric species which are separated by a further atom, and hydroxyl groups in a 1,4 relationship are attached to atoms in the polyhydric species which are separated by a further two atoms.

To facilitate the interaction of the two hydroxyl groups with the boronic acid species, it may be preferable that the hydroxyl groups are cis to each other. Hydroxyl groups in a cis relationship with each other include those which are positioned on the same side of a reference plane in the polyhydric species. For example, they could be located on the same face of a ring which forms part of the polyhydric species.

In some embodiments of the invention, the polyhydric species is a carbohydrate containing species. Carbohydrate containing species include species having moieties which contain carbon, oxygen and hydrogen atoms, such as saccharide moieties. For example, the species may contain moieties having the general formula Cₓ(H₂O)_{y}. Also included are moieties which are the deoxy forms of moieties having the general formula Cₓ(H₂O)_{y}, such as 2-deoxy-D-ribose, or oxidised forms of moieties having the general formula Cₓ(H₂O)_{y}, such as gluconolactone.

Carbohydrates are components of nucleosides, nucleotides, RNA and DNA, glycoproteins, glycolipids and glycosaminoglycans, and accordingly carbohydrate containing species include these species.

Carbohydrate containing species also include monosaccharides, oligosaccharides and polysaccharides.

In some preferred embodiments, the polyhydric species is selected from posttranslationally modified peptides, polypeptides and proteins; glycated DNA (or RNA), glycated lipids, and mono-, oligo- and poly-saccharides.

In some embodiments, the polyhydric species is a phosphate containing species. Phosphate containing species includes species having the moiety —O—P(O)(OH)₂, irrespective of its state of ionisation.

The polyhydric species may be the product of posttranslational modification of polypeptides, as many types of such modification include hydroxyl groups that are capable of interaction with boronic acid species disclosed herein. Posttranslational modification of polypeptides and proteins is discussed in more detail below.

The methods of the present invention may also be useful in identifying proteins that bind sugar molecules. Accordingly, polyhydric species include proteins bound to sugar molecules by covalent, ionic and other non-covalent interactions such as hydrogen bonding.

As discussed above, different polyhydric species may migrate through the gel at different speeds in the methods of the invention. They may migrate through the gel at different speeds according to their mass/charge ratio and/or their boron affinity.

Posttranslational Modification

The present invention may also be used for the detection of post-translational modification of peptides, polypeptides and proteins, and in particular the detection and identification of glycated species. Many types of posttranslational modification involve the covalent attachment of moieties comprising hydroxyl groups that are capable of interaction with boronic acid species disclosed herein.

Posttranslational modification includes chemical modification of amino acids and the attachment of biochemical functional groups after their incorporation into polypeptides, during protein synthesis. This can, for example, have the effect of extending the range of function of proteins. Posttranslational modifications can control a protein's localization, turnover and active state structural changes and also manipulate their three-dimensional structure and interactions with other proteins. The analysis of these modifications is key to understanding the structure and function of proteins and protein-protein interactions. Accordingly, methods which allow the detection, characterisation and monitoring of posttranslational modifications will be of clear benefit to the study of protein structure and behaviour.

Undesired posttranslational modifications also may occur, for example, in the form of oxidation and glycation, the non-enzymatic attachment of sugars to proteins. Glycation is known as a biomarker for ageing and disease states related to diabetic complications^{6,23,24} . The oxidised glucose derivative δ-gluconolactone, for instance, has been shown to cause glycation of hemoglobin, which may be a factor in the vascular complications of diabetes ^{25,26}. The accumulation of δ-gluconolactone could play also play in important role in ageing processes²⁷ (see also²⁸). Accordingly, methods which allow the monitoring and detection of posttranslational modifications may be useful in monitoring and/or diagnosis of diseases, conditions or biological processes.

Posttranslational modification also occurs in peptides, polypeptides and proteins expressed recombinantly. The posttranslational modification of recombinantly produced peptides, polypeptides and proteins may be different from the posttranslational modification of the same peptides, polypeptides and proteins when produced in native conditions (i.e. when produced by the organism which naturally produces the peptide). It is therefore highly desirable to be able to monitor and control posttranslational modification of recombinantly expressed peptides, polypeptides and proteins. Accordingly, methods which allow the detection, characterisation and monitoring of posttranslational modification of peptides, polypeptides and proteins will be of clear benefit to technologies involving recombinant expression, as will methods for the resolving and separating posttranslationally modified peptides, polypeptides and proteins. For example, control of post-translational gluconoylation in recombinant proteins is significant in the production of proteins of pharmaceutical and medical applications²⁹.

As discussed above, in many cases posttranslational modification of polypeptides and proteins may involve the introduction of moieties comprising a plurality of hydroxyl groups. Accordingly, polyhydric species include posttranslationally modified peptides, polypeptides and proteins, wherein the posttranslational modification may involve the introduction of a moiety comprising a plurality of hydroxyl groups. Introduction of a moiety by posttranslational modification includes covalent attachment of the moiety to the peptide, polypeptide or protein being modified. The post-translationally modified peptide, polypeptide or protein may have been modified by the addition of carbohydrate components, for example by glycation, glycosylation or gluconoylation. Alternatively or additionally, the peptide, polypeptide or protein may have been modified by phosphorylation.

Accordingly, the polyhydric species of the present invention include glycated polypeptides and proteins, gluconoylated polypeptides and proteins, lactosyl polypeptides and proteins, phosphorylated polypeptides and proteins and glycosylated polypeptides and proteins.

The examples below show that the methods disclosed herein can be used to resolve, separate and detect glycation products such as δ-gluconolactone, as well as glycosylated and phosphorylated proteins.

Specific examples of posttranslational modification include, for example, spontaneous α-N-6-Phosphogluconoylation. This has been observed and described in recombinantly expressed proteins fused to a histidine affinity tag³⁰⁻³². 6-phosphategluconlactone (6PGL) is an intermediate of the pentose phosphate pathway, which is produced by glucose-6-phosphate dehydrogenase (G6PD), and is a potent electrophile which reacts with the N-terminal amino group of histidine-tagged protein forming amine-linked product with the protein³². This modification has been shown to adversely affect protein activity³³ and interferes with crystallization of proteins³⁴. It may also impair structure or immunogenicity of the expressed protein, which would greatly obstruct the use of recombinantly produced histidine-tagged proteins in research, diagnostics and therapy. As a model for analysing this modification, a protein construct based on Staphylococcus aureus immune-subversion protein Sbi may be used. This protein has been shown to inhibit the innate immune system³⁵ and is currently being developed as a therapeutic for complement-mediated acute inflammatory diseases. The Sbi-III-IV construct has a 25-residue N-terminal tag with sequence MSYHHHHHHDYDIPTTENLYFQGAM and mass spectrometry analysis of similar constructs containing this tag have shown that this sequence is specifically prone to 6-phosphogluconoylation. In the past, this undesired N-terminal adduct could only be detected by mass spectrometric analysis of the protein. The methods of the present invention may provide improved methods of detecting and separating peptides, polypeptides and proteins which have been subject to spontaneous α-N-6-Phosphogluconoylation.

Another example of posttranslational modification which introduces a moiety comprising a plurality of hydroxyl groups is formation of advanced glycation end products (AGEs), which starts with non-enzymatic addition of a sugar or a sugar-fragmentation product to a protein, followed by rearrangement to a linear Schiff-base adduct, finally rearranging to a protein-bound Amadori product. In later stages of the glycation process AGEs are formed, which may include a broad range of heterogeneous fluorescent and yellow-brown products, including nitrogen-containing and oxygen-containing heterocycles, resulting from subsequent oxidation and dehydration reactions^{36,37}. It will be understood that the methods of the present invention may be used to resolve, separate monitor or detect one or more of the stages of the formation of AGEs described above, as each stage may involve the introduction or modification of moieties containing a plurality of hydroxyl groups.

AGEs are implicated in certain diseases and conditions, and may be markers of these diseases or conditions. Additionally, AGEs may prove to be markers or indicators useful in monitoring biological processes such as ageing. As an example, β-amyloid deposits, the hallmarks of Alzheimer's disease, contain sugar-derived AGEs. Accordingly, the methods of the present invention may be useful in monitoring and detecting AGEs as markers associated with diseases, conditions and biological processes, or in monitoring and diagnosing diseases or conditions associated with AGEs. The methods may also prove useful in designing new inhibitors and/or drugs which can control, reduce or prevent the formation of AGEs, for example inhibitors of -amyloid formation and drugs for treating Alzheimer's disease.

New methods for the analysis of posttranslational modifications may lead to better understanding of the process of posttranslational modification, which understanding may prove valuable in medical applications. For example, it has been found that β-amyloid deposits contain copper ions in addition to sugar-derived AGEs. It has also been shown in vitro that the formation of covalently cross-linked high-molecular-mass β-amyloid peptide oligomers, using synthetic β-amyloid peptide and glucose or fructose, is accelerated by micromolar amounts of copper (and iron) ions³⁸. This finding may explain the specific formation of δ-gluconolactone adducts to N-terminal histidine metal-affinity tags in recombinant proteins, suggesting that histidine tag-bound metal ions could be involved in the acceleration of this process as well.

Applications

The materials and methods disclosed herein are well suited to separating samples containing species capable of reversibly interacting with boron.

The methods disclosed herein may be used for detecting markers linked to diseases and conditions where the markers contain functional groups that are capable of reversible interaction with the boronic acid groups present in the gel. Markers which may be detected by the methods of the present invention include disease linked carbohydrates in the blood which can be indicative for example of cancer. Other cancer markers include the CA-125 antigen and heptasaccharide markers. Glycated proteins, including early stage glycated proteins can be indicative of diseases, conditions or biological processes including ageing, diabetes, Alzheimer's disease. Polyhydric species such as carbohydrates and posttranslationally modified peptides can also be markers for microbial infections.

### EXPERIMENTAL EXAMPLES

Methods

Human Sera

Human serum was obtained from Lonza, and serum albumin from Sigma-Aldrich. In vitro glycated human serum was obtained by incubating human serum with 50 mM glucose, fructose, mannose, maltose, galactose or sucrose (in the presence of 0.1% azide), in a dry heating block at 37° C. for 7 or 10 days under aseptic conditions. Type 1 diabetes human sera (from patients diagnosed using glutamic acid dehydrogenase (GAD) and insulin autoimmune antibodies (IAA and IA-2)) were obtained from SunnyLab UK.

TASTPM Mouse Brain Homogenates

Cortex from heterozygote transgenic mouse over-expressing hAPP695swe and presenilin-1 M146V mutations (TASTPM) were obtained from GlaxoSmithKline, along with age-matched wild type C57BL/6, as previously reported^{39,40}. All animal studies were ethically reviewed and carried out in accordance with Animals (Scientific Procedures) Act 1986 and the GSK Policy on the Care, Welfare and Treatment of Animals. The cortex samples were mechanically homogenised in 0.5 mL 50 mM Tris-EDTA buffer pH 7.4 with protease inhibitor (Roche), and spun at 2,000 g for five minutes to obtain crude homogenates.

Manduca Sexta Hemolymph

Manduca sexta larvae were kept individually on a wheat germ based artificial diet at 25° C. with 17 h light: 7 h dark photoperiod. The larvae were chilled on ice for 30 minutes, and bled in sterile tubes after brief sterilisation with 70% ethanol and cutting the ‘tail’ near the tip. 5 μL of saturated phenoloxidase inhibitor 1-phenyl-2-thiourea (PTU, approx. 20 mM PTU in PBS) were added to 300 μL of hemolymph to prevent melanisation.

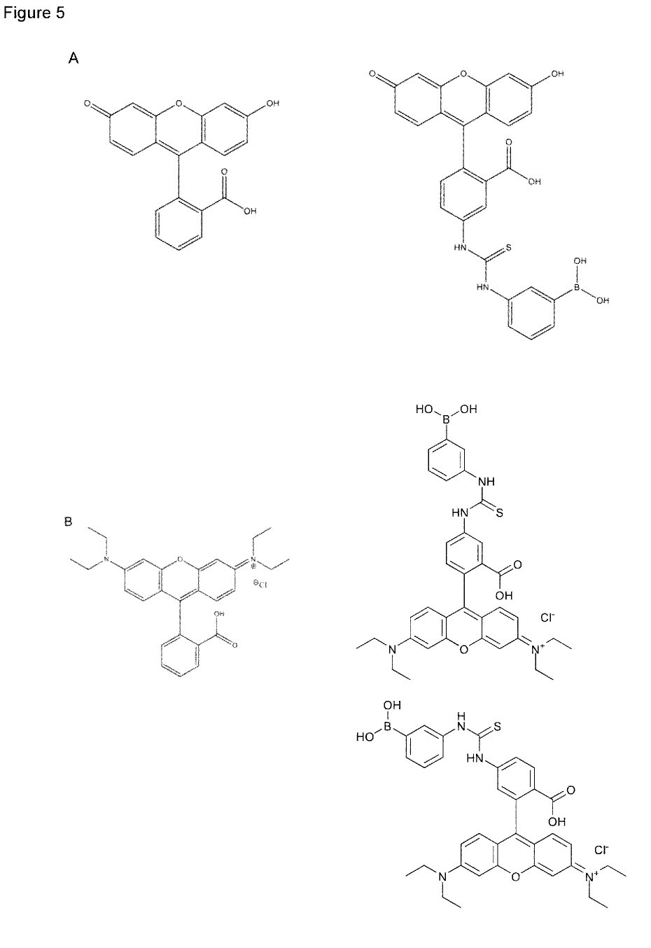

Synthesis of fluorescent boronic acid compounds Fluorescein-boronic acid (3-(3-(3′,6′-Dihydroxy-3-oxo-3H-spiro[isobenzofuran-1,9′-xanthene]-5-yl)thioureido)phenylboronic acid, structure shown in FIG. 5A right) was synthesised as previously reported⁴¹. Briefly, 3-aminobenzeneboronic acid (0.35 g, 2.57 mmol) was added to a solution of fluorescein isothiocyanate (1.00 g, 2.57 mmol) in DMF (5 mL), stirred at room temperature for 12 h then poured into methanol (10 mL). The solvents were removed in vacuo and residue re-dissolved in the minimum amount of fresh methanol. Chloroform was added and the product was obtained as a bright orange precipitate (920 mg, 68% yield).

Rhodamine-boronic acid (structure shown in FIG. 5B right) was synthesised as follows. Triethylamine (3 eq. 0.56 mmol, 80 μL) was added to a solution of Rhodamine B isothiocyanate (100 mg, 0.18 mmol) and 3-aminobenzene boronic acid (1.1 eq, 0.21 mmol, 28 mg) in dry DMF (5 mL) under N₂. The reaction mixture was stirred overnight, then the solvent removed in vacuo. The reaction mixture was passed through a short pad of silica eluting with DCM, DCM/methanol (1% to 10%). The solvent was removed to give the product as a dark purple-red solid (65 mg, 58% yield).

Labelling and Electrophoresis of Glycated Proteins with Fluorescent Boronic Acids

1 μL of 10-fold diluted human serum was incubated with 0.5 mM fluorescent boronic acid at room temperature for 1 h. Less stable samples can be labelled overnight at 4° C. Control samples were labelled with Fluorescein (Sigma-Aldrich; structure shown in FIG. 5A left) or Rhodamine B (Sigma-Aldrich; structure shown in FIG. 5B left).

Gel electrophoresis was performed using Xcell surelock mini-cell (Invitrogen) and Power Pac 300 (Bio-Rad). Proteins were blotted onto a 0.45 μm Immobilon-P PVDF membrane (Millipore) in transfer buffer (25 mM tris, 192 mM glycine) at 15 V for 50 minutes, using a Trans blot SD Semi-dry transfer cell (Bio-Rad). The gels were visualised prior to protein staining with UV light (AlphaImager 3400 gel imaging system, Alpha Innotech; wavelength 365nm, with orange (595 nm) or green (537 nm) filters) or Dark Reader® (Clare Chemicals Research Inc.; wavelength range 420-520 nm, with amber (-530 nm) filter). Contrast has been optimised for the gel and blot images.

After blotting, the membrane was blocked using TBST buffer (50 mM Tris, 150 mM NaCl, 0.05% Tween 20, pH 7.6) with 5% milk powder at room temperature for 1 h. HRP-conjugated anti-AGE monoclonal antibody (1:800 dilution) was then applied to the blot at room temperature for 1 h. After five 30 min washes, the blot was incubated with peroxidase substrate for enhanced chemiluminescence (ECL, Pierce) at room temperature for 1 min before developing on film. The monoclonal anti-AGE antibody was anti-AGE-BSA mAb clone 6D12, Cosmo Bio Co Ltd.).

Results

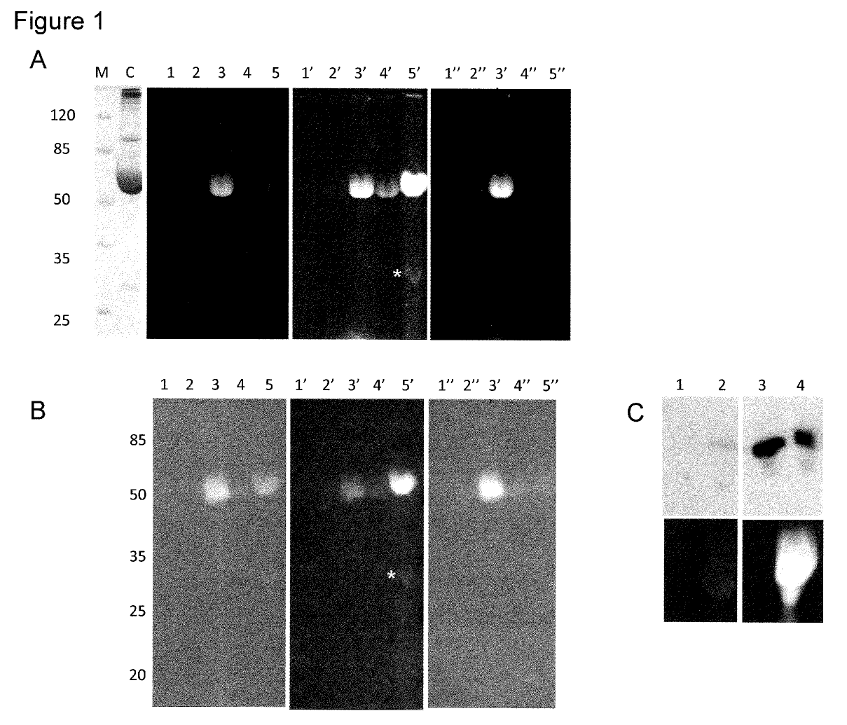

Glycated proteins present in normal human serum were visualised after incubation of serum with fluorophore-appended boronic acids in SDS-PAGE (Flu-PAGE) and Eastern blotting (Flu-BLOT) (FIG. 1). Incubation of normal human serum with fluorescein, fluorescein-boronic acid, rhodamine and rhodamine-boronic acid (FIG. 1A; structures of these compounds are shown in FIG. 5) resulted in specific fluorescent staining of glycated HSA in SDS-PAGE, only in samples containing fluorescein- or rhodamine-appended boronic acid. No known glycosylated or non-glycosylated serum proteins were labelled using this method indicating that, similar to mP-AGE, the fluorescent boronic acids used in Flu-PAGE specifically interact with fructosamine modified proteins. Glycated proteins labelled with both fluorophores showed identical electrophoretic migration properties and were readily transferred from the gels after electrophoresis (shown in FIG. 1A) to a polyvinyl difluoride (PVDF) membrane and directly visualised with visible blue and UV light (FIG. 1B). This Flu-BLOT method can be used in conjunction with standard Western blotting methods, as shown in FIG. 1C. MALDI-TOF analysis of an additional fluorescent protein band in the rhodamine-boronate labelled human serum, observed in both Flu-PAGE and Flu-BLOT (FIGS. 1A and 1B, lane 5′, indicated by a white asterisk) was identified as Apolipoprotein A-I (ApoA-I).

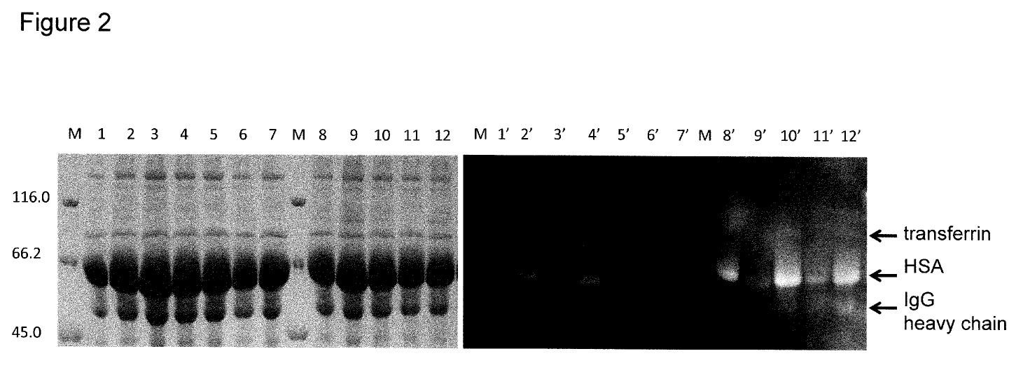

To demonstrate that the Flu-PAGE method can be used to follow the glycation of human serum proteins over time, normal human serum was incubated with various reducing sugars, including glucose, fructose, mannose, maltose, and galactose. After 7 days of incubation with these sugars the HSA bands clearly showed increased fluorescence intensity in Flu-PAGE in the presence of fluorescein-boronic acid, especially in the sera that had been incubated with glucose and mannose (FIG. 2). After 10 days of incubation, a significant increase in fluorescein-boronic acid labelling of HSA was observed in the serum samples incubated with glucose, mannose and galactose, indicating increased levels of glycation by these sugars. A variety of other fluorescent protein bands were observed, two of which were identified as serotransferrin and Ig γ chain constant region, using MALDI-TOF MS. These results are in agreement with the fluorescein-boronic acid labelling of sera from type I diabetes patients (FIG. 7), showing a clear increase in fluorescence intensity of HSA in two of the diabetic sera. One of the diabetic sera displayed a level of HSA fluorescence comparable to control serum. However, this sample showed a distinct additional fluorescent band at the 50 kDa position in the gel, indicative of IgG heavy chain glycation.

The possible use of fluorescent boronic acid labelling of glycated proteins as a proteomics tool in 1) an insect model system that is currently in development as an animal model for diabetes (Manduca sexta) and 2) a murine model for AD, was also tested.

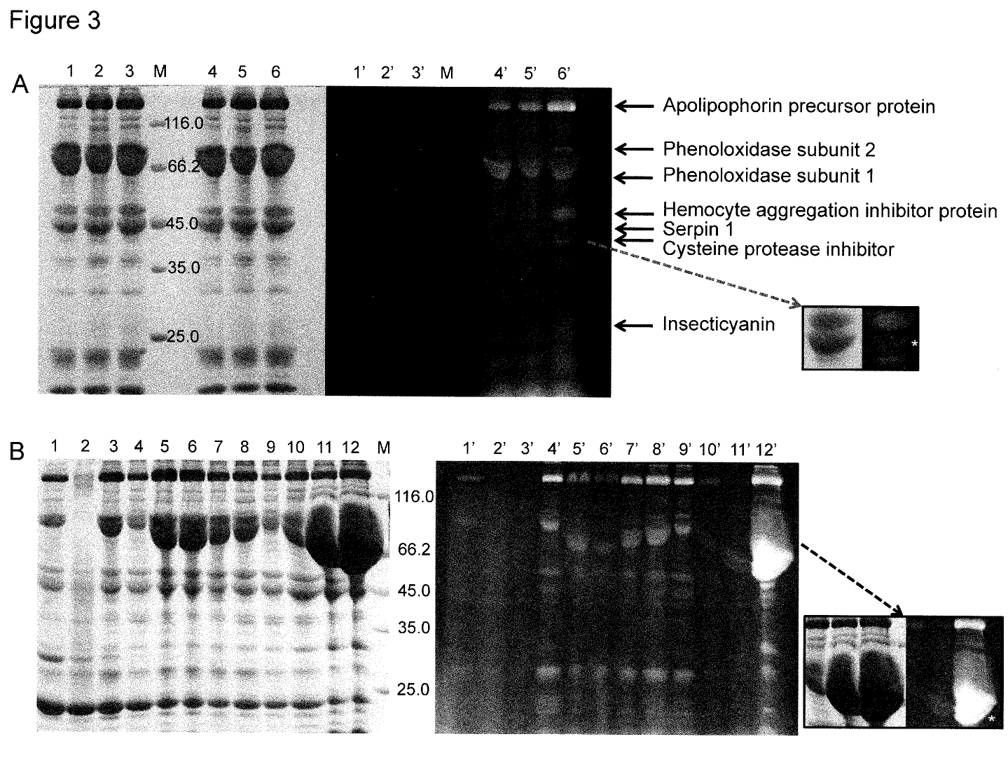

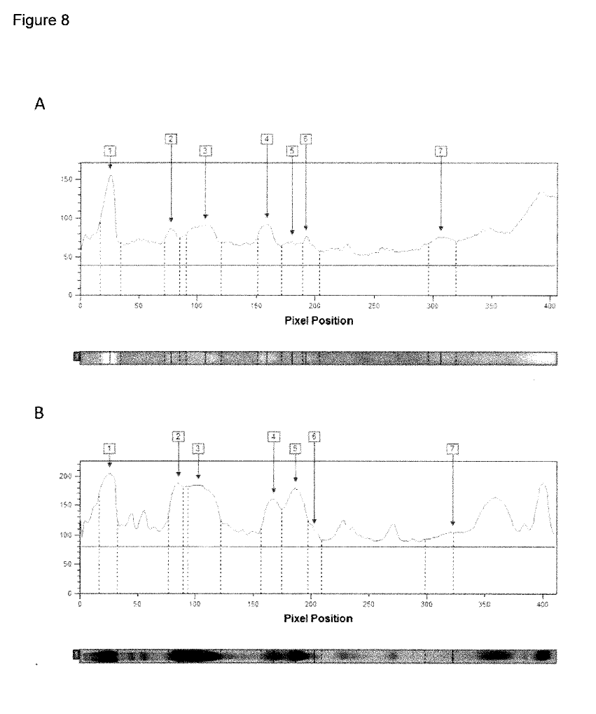

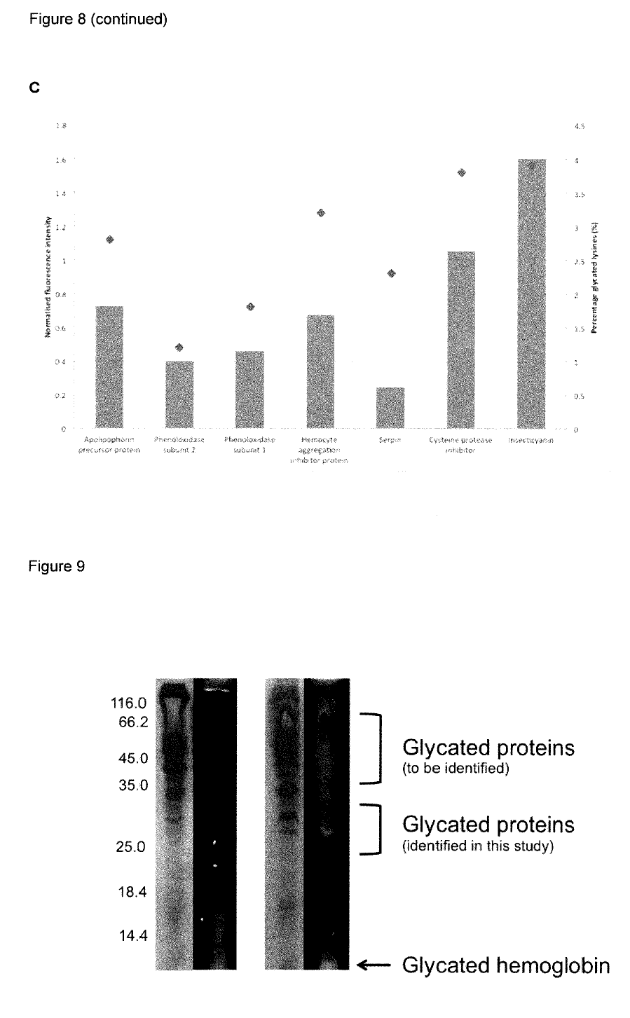

Insects are becoming increasingly popular as protein glycation model systems and have been applied for the identification of biomarkers in ageing, in Drosophila⁴², and for the testing anti-diabetic drugs, in a hyperglycemic silk worm model²³. Incubation of Manduca sexta hemolymph with fluorescein-boronic acid resulted in labelling of at least 10 different protein bands in Flu-PAGE (FIG. 3A). It is interesting to note that the majority of protein bands seen in the Coomassie stained gel were also labelled in the Flu-PAGE analysis, indicating that a large majority of the Manduca sexta hemolymph proteins are glycated. However, not all distinct fluorescent bands correspond to a high level of protein staining, signifying that some highly glycated proteins are present in low concentrations in the hemolymph samples. Conversely, some highly expressed proteins showed low levels of glycation (as indicated in FIGS. 3A and 3B, insets; see also FIG. 8). Six protein bands, with approximate molecular weights: >200, 80, 45, 40 and 25 kDa were analysed by MALDI-TOF and identified using the Manduca sexta proteome database (Agricultural Pest Genomics Resource Database: www.agripestbase.org). The most prominent fluorescent protein band observed in the gels, indicating high levels of glycation, corresponds to Apolipophorin precursor protein a lysine- and arginine-rich 367 kDa protein (see Table 1 below) that constitutes the major component of lipophorin, which mediates transport for various types of lipids in hemolymph. Other glycated proteins identified in Manduca hemolymph include: phenoloxidase subunits 1 and 2, two copper-containing oxidases (79 and 80 kDa, respectively) that function in the formation of pigments such as melanins and other polyphenolic compounds; hemocyte aggregation inhibitor protein precursor (48 kDa); putative CiA cysteine protease precursor (38 kDa) and insecticyanin, a 23 kDa protein synthesised in the caterpillar epidermis and secreted into the hemolymph.

The Flu-PAGE method was also used to follow protein glycation during the development of the Manduca sexta caterpillar. The results in FIG. 1C show that whilst the levels of most hemolymph proteins remains constant during development, concentrations of proteins such as phenoloxidase vary dramatically and its expression appears to follow a wave pattern, peaking just before pupation. Glycation levels of the protein appear to be much lower at the beginning of an expression wave and much higher at the end of a wave, especially when expression levels peak before reaching pupation. A very striking example of this can be observed in the case of phenoloxidase subunit 1. Whilst a significant increase in the level of expression of this protein can be observed in the Coomassie stained gel on day 17 (FIG. 3B, lane 11), hardly any fluorescence was detected in the Flu-PAGE analysis. On day 18, just before entering pupation, the protein clearly became glycated as evidenced by the dramatic increase in fluorescence in the Flu-PAGE gel.

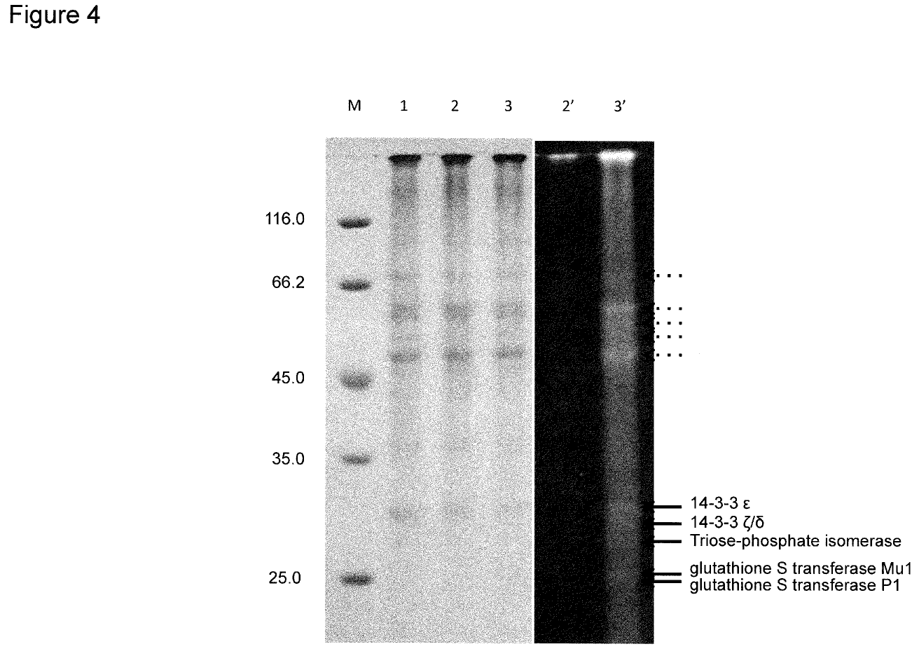

For the analysis of protein glycation in an AD mouse model, brain homogenates from transgenic mice that develop extensive amyloid β (Aβ) plaque pathology and normal controls were used. Brain homogenates were prepared from the cortices of 5-month-old heterozygote transgenic mice overexpressing both the hAPP695swe and the presenilin-1 M146V mutations (TASTPM ⁴⁴), and from aged matched wild type C57BL/6 control animals. The results from the Flu-PAGE analysis of the brain samples are shown in FIG. 4 and FIG. 9, where a UV image of the SDS-PAGE gel is compared with normal Coomassie stained gel. Although many bands are visible in the Coomassie stained gel, no other distinct fluorescent bands can be observed in the control cortex sample, with the exception of a strong band at approximately 120 kDa and a weaker low molecular weight band of about 15 kDa. In the TASTPM sample a large number of distinct fluorescent bands can be observed. Two groups of the prominent bands, can be seen between the 66 and 45 kDa molecular weight markers and between 35 and 25 kDa (FIG. 4). Five fluorescent bands between 35 and 25 kDa that are absent in the control mouse cortex sample were identified by MALDI-TOF analysis as 14-3-3 proteins (ε and ζ/δ, with respective masses of 29 and 28 kDa), triosephosphate isomerase (27 kDa), glutathione S-transferases Mul (26 kDa) and P1 (23 kDa), respectively.

Discussion

Glycated proteins are important biomarkers for age-related disorders, such as diabetes, cardiovascular diseases, autoimmune diseases, cancer, and AD. Biomarkers identifying biological and physiological entities associated with such diseases are becoming increasingly important for drug discovery. The present inventors have shown that glycated proteins can be visualised and identified in a variety of complex biological samples, including human serum, Manduca sexta hemolymph and mouse brain cortex homogenates, using fluorescent boronic acids in Flu-PAGE and Flu-BLOT.

In samples of human serum, specific labelling of glycated HSA was observed in Flu-PAGE and Flu-BLOT and detection was most effective with fluorescein-boronic acid as it resulted in specific labelling of the glycated HSA band with all the wavelengths and filter combinations used (conventional visible blue light with 530 nm filter, and UV light sources with 595 and 537 nm filters). Glycated proteins labelled with rhodamine-boronic acid could be visualised best using UV with a 595 nm (orange) filter, however some aspecific labelling of HSA was observed in the rhodamine control lane. At this wavelength another glycated protein band was observed in the rhodamine-boronic acid labelled serum sample (FIG. 1A). The identified ApoA-I protein plays an important role in the transport of phospholipids and free cholesterol to the liver and its glycation has been linked to atherosclerosis and shown to contribute to its impaired action⁴⁵. Analysis of human serum samples incubated with different reducing saccharides (FIG. 2), using Flu-PAGE, further identified serum proteins serotransferrin and IgG heavy chain as glycation targets. All of these high-abundance plasma proteins are well-known Amadori-modified proteins in type 2 diabetes and are linked with vascular complications^{46,47}.

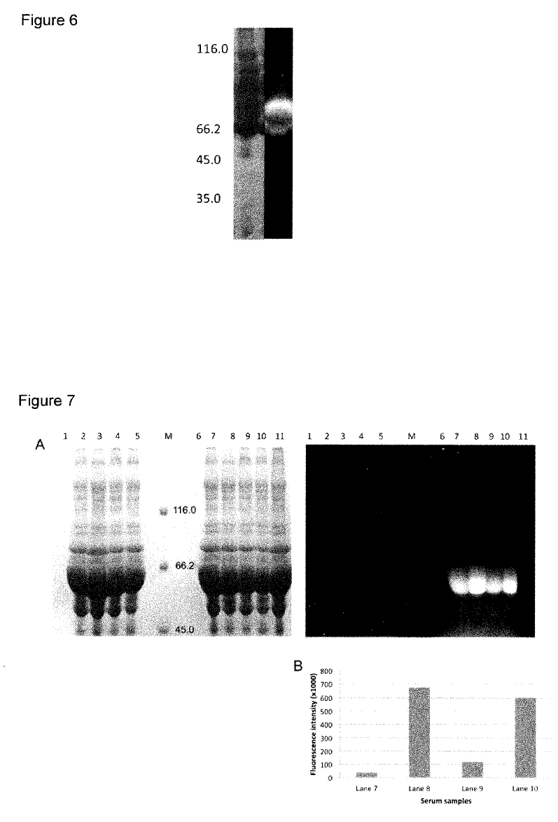

The Flu-PAGE results observed with saccharide-incubated serum samples are consistent with those found when analysing HSA glycation using mP-AGE²⁰, showing strongest interactions between boronic acid and cis-1,2-diol-containing fructosamine adducts (resulting from glycation with glucose, mannose and galactose), stabilised by an electrostatic interaction between the protonated amino group and the negatively charged boronate moiety. When using Flu-PAGE in conjunction with mP-AGE analysis of the serum sample, the highest fluorescence intensity can be observed in the glycated HSA fraction that is retained in its electrophoretic mobility via interaction with the gel-incorporated MPBA (FIG. 6).

Many proteins in Manduca sexta hemolymph appear to be affected by glycation in Flu-PAGE analysis. The most prominent fluorescent protein band corresponds to Apolipophorin precursor protein, which has a function similar to Apo A-I, identified as a glycated protein in human serum (FIG. 1A), indicating that Manduca sexta could also be a useful model system for diseases such as atherosclerosis and AD. Some of the fluorescent bands in the hemolymph Flu-PAGE pattern do not correspond to a clear Coomassie stained protein band, implying that some highly glycated proteins are present in low concentrations. During the development of Manduca sexta the levels of most hemolymph proteins remain constant in all larval stages (FIG. 1C), however, concentrations of proteins such as phenoloxidase vary dramatically between larval stages and its expression appears to follow a wave pattern, peaking just before pupation. Flu-PAGE analysis showed that levels of protein glycation appeared to be very low during the early stages of development and increase towards later stages. Interestingly, the fluorescein-boronic acid labelling of phenoloxidase also followed a wave-like pattern that is out of phase with its expression levels. The glycation of this protein appears to reach its peak in the days close to pupation. This corresponds with high hemolymph glucose levels at this stage of development (16 mM) when compared to with feeding larvae (1 mM⁴⁸, making Manduca sexta an ideal model for following the effects of glycation during a hyperglycemic episode.

With this example, the inventors have shown that fluorescent boronic acid gel electrophoresis can be used to specifically identify glycated proteins in complex samples for normal development in non-disease states and in concert follow their temporal expression and glycation patterns. It should be noted that these insects were reared on a very high sugar diet, which may explain the overall high levels of glycation seen in their blood relative to the mammalian samples. Remarkably, there is a clear correlation between fluorescence intensity of the identified hemolymph proteins and the percentage of predicted glycated lysines in these proteins (FIG. 8, also see Table 1).

Similar to the Manduca sexta hemolymph samples, some of the distinct fluorescent bands seen in the Flu-PAGE analysis of TASTPM cortex homogenates do not show a clear protein band in corresponding the Coomassie stained gels, indicating that these proteins are present in low concentrations in the brain samples. MALDI-TOF analysis of the low molecular weight band (˜15 kDa) present in both control and TASTPM homogenates identified this protein as hemoglobin, a well-known glycation marker in diabetes. Although the hemoglobin band is present in both control and TASTPM homogenates it appeared brighter in the transgenic samples, indicating a higher level of glycation (FIG. 9). The identified 14-3-3 ε and ζ/δ proteins have previously been linked with AD and shown to be present in the neurofibrillary tangles of AD brains⁴⁹. It is interesting to note that of all the glycated proteins identified in this study, 14-3-3 ζ/δ has the highest percentage of predicted glycated lysines (Table 1). Both of the GST proteins, also discovered in the Flu-PAGE analysis of the TASTPM cortex homogenates, belong to a class of enzymes that function in the detoxification of hydrophobic electrophilic compounds, including carcinogens, therapeutic drugs, environmental toxins and products of oxidative stress, by conjugation with reduced glutathione. Glycation of these enzymes could affect their function and increase susceptibility to environmental toxins and carcinogens. Oxidative stress is strongly implicated in the pathology of AD and a recent study has shown that pro-oxidant conditions increase amyloidogenic processing of amyloid precursor protein (APP) and therefore might contribute to disease progression⁵⁰.

In conclusion the present inventors have shown that fluorescent boronic acids can be used in gel electrophoresis and Eastern blotting for the detection and identification of individual glycated proteins in complex samples.

The advantages of the Flu-PAGE and Flu-BLOT methods include:
Direct visualisation of glycated proteins. Flu-PAGE and Flu-BLOT are the only methods that can directly visualise glycated proteins in SDS-PAGE and blots. This labelling method does not affect the electrophoretic migration properties of the proteins, nor does it hinder their subsequent identification by MS.
Detection of glycated proteins in complex samples. This method is unique in that it detects glycated proteins in complex protein mixtures without the need of additional enrichment or purification techniques such as BAC. This method also enables the study of glycated proteins in the context of other non-affected proteins in fluid (human serum and insect hemolymph) and solid (mouse brain cortex homogenates) biosamples.
Detection for early glycation adducts. The fluorescent boronic acids used in Flu-PAGE specifically interact with fructosamines, enabling the detection of early glycation adducts. With current methods to analyse glycated proteins using boronic acids (such as BAC) all types of glycation modifications in a sample are retained, including N- and O-linked glycans. Anti-AGE antibodies, used to detect glycated protein in western blotting methods, only recognise late stage glycation end products. Most of these antibodies lack specificity because they have been raised against a mixture of AGEs.
Flu-PAGE and Flu-Blot can be used in conjunction with other protein analysis tools. Flu-PAGE can be used in combination with an earlier developed method (mP-AGE) to label and separate glycated from non-glycated proteins. Flu-Blot can also be used in conjunction with antibody detection in western blots to enable multiple labelling of proteins.

Using a combination of Flu-PAGE and Flu-Blot the present inventors have i) identified differences in glycation between normal human serum and that of patients suffering from type 1 diabetes, ii) discovered novel glycated proteins in hemolymph from Manduca sexta, a new animal model system for diabetes, and iii) unveiled proteins that are affected by glycation in an AD animal model. This easy-to-use method enables the study of protein glycation patterns in complex samples over time and in the context of development and disease. These results signify that fluorescent boronic acid gel electrophoresis (Flu-PAGE) and Eastern blotting (Flu-BLOT) provide a powerful and cost-effective first-stage proteomics tool for the identification and analysis of glycated protein biomarkers in ageing and age-related diseases and for probing novel glycation inhibitors and other anti-AGE therapies.

TABLE 1
Glycated proteins identified by Flu-PAGE.

| | | | | | Number |
|---|---|---|---|---|---|
| | | | | | of |
| | | | Number | | glycated |
| | | | of | Number of | K/total |
| | Database | MW | amino | K/glycated | amino |
| Protein | number | (kDa) | acids | K* | acids (%) |
| | Manduca sexta hemolymph | | | | |
| apolipophorin | AAB53254.1 | 367 | 3305 | 282/92 (33%) | 2.8 |
| precursor | | | | | |
| protein | | | | | |
| pro- | Q25519.3 | 80 | 695 | 31/8 (26%) | 1.2 |
| phenoloxidase | | | | | |
| subunit 2 | | | | | |
| pro- | O44249 | 79 | 685 | 27/12 (44%) | 1.8 |
| phenoloxidase | | | | | |
| subunit 1 | | | | | |
| hemocyte | ACW82749.1 | 48 | 434 | 28/14 (50%) | 3.2 |
| aggregation | | | | | |
| inhibitor | | | | | |
| protein | | | | | |
| precursor | | | | | |
| serpin 1 | AAC47343.1 | 43 | 392 | 34/9 (26%) | 2.3 |
| putative C1A | CAX16635.1 | 38 | 342 | 29/13 (45%) | 3.8 |
| cysteine | | | | | |
| protease | | | | | |
| precursor | | | | | |
| insecticyanin | CAA45969.1 | 23 | 206 | 19/9 (47%) | 4.4 |
| | TASTPM cortex homogenates | | | | |
| 14-3-3 ε | P62259 | 29 | 255 | 18/3 (17%) | 1.2 |
| 14-3-3 ζ/δ | P63101 | 28 | 245 | 20/11 (55%) | 4.5 |
| Triose | CAA37420.1 | 27 | 249 | 20/5 (25%) | 2.0 |
| phosphate | | | | | |
| isomerase | | | | | |
| glutathione | P10649 | 26 | 218 | 18/4 (22%) | 1.8 |
| S-transferase | | | | | |
| Mu1 | | | | | |
| glutathione | P19157 | 24 | 210 | 12/6 (50%) | 2.9 |
| S-transferase | | | | | |
| P1 | | | | | |
| Hemoglobin | | | | | |
| α | P01942.2 | 15 | 142 | 22/11 (50%) | 7.7 |
| β | P02088.2 | 16 | 147 | | |
| | Human serum | | | | |
| serotransferrin | P02787.3 | 77 | 698 | 58/18 (31%) | 2.6 |
| serum albumin | P02768 | 69 | 609 | 60/24 (40%) | 3.9 |
| IgG heavy | AAA02914.1 | 52 | 476 | 32/18 (56%) | 3.8 |
| chain | | | | | |
| apolipoprotein | P02647.1 | 31 | 267 | 22/9 (41%) | 3.4 |
| A-I | | | | | |

*Analysis and prediction of mammalian protein glycation. Johansen M B, Kiemer L and Brunak S, Glycobiology, 16:844-853, 2006.

### REFERENCES

All documents mentioned in this specification are incorporated herein by reference in their entirety.

1. Vlassara, H. Advanced glycation in health and disease: role of the modern environment. Ann N Y Acad Sci 1043, 452-60 (2005).

2. Vlassara, H. & Uribarri, J. Glycoxidation and diabetic complications: modern lessons and a warning? Rev Endocr Metab Disord 5, 181-8 (2004).

3. Kurien, B. T. & Scofield, R. H. Autoimmunity and oxidatively modified autoantigens. Autoimmun Rev 7, 567-73 (2008).

4. Wells-Knecht, M. C., Thorpe, S. R. & Baynes, J. W. Pathways of formation of glycoxidation products during glycation of collagen. Biochemistry 34, 15134-41 (1995).

5. Rahbar, S. Novel inhibitors of glycation and AGE formation. Cell Biochem Biophys 48, 147-57 (2007).

6. Unoki, H. & Yamagishi, S. Advanced glycation end products and insulin resistance. Curr Pharm Des 14, 987-9 (2008).

7. Vitek, M. P. et al. Advanced glycation end products contribute to amyloidosis in Alzheimer disease. Proc Natl Acad Sci USA 91, 4766-70 (1994).

8. van Heijst, J. W., Niessen, H. W., Hoekman, K. & Schalkwijk, C.G. Advanced glycation end products in human cancer tissues: detection of Nepsilon-(carboxymethyl)lysine and argpyrimidine. Ann NY Acad Sci 1043, 725-33 (2005).

9. Smith, M. A., Monnier, V. M., Sayre, L. M. & Perry, G. Amyloidosis, advanced glycation end products and Alzheimer disease. Neuroreport 6, 1595-6 (1995).

10. Smith, M. A., Sayre, L. M., Monnier, V. M. & Perry, G. Oxidative posttranslational modifications in Alzheimer disease. A possible pathogenic role in the formation of senile plaques and neurofibrillary tangles. Mol Chem Neuropathol 28, 41-8 (1996).

11. Smith, M. A. et al. Advanced Maillard reaction end products are associated with Alzheimer disease pathology.Proc Natl Acad Sci U S A 91, 5710-4 (1994).

12. Klenk, D.C. et al. Determination of glycosylated hemoglobin by affinity chromatography: comparison with colorimetric and ion-exchange methods, and effects of common interferences. Clin Chem 28, 2088-94 (1982).

13. Zhang, Q., Ames, J. M., Smith, R. D., Baynes, J. W. & Metz, T. O. A Perspective on the Maillard Reaction and the Analysis of Protein Glycation by Mass Spectrometry: Probing the Pathogenesis of Chronic Disease. Journal of Proteome Research 8, 754-769 (2008).

14. Zhang, Q. et al. Enrichment and analysis of nonenzymatically glycated peptides: boronate affinity chromatography coupled with electron-transfer dissociation mass spectrometry. J Proteome Res 6, 2323-30 (2007).

15. Montgomery, H., Tanaka, K. & Belgacem, O. Glycation pattern of peptides condensed with maltose, lactose and glucose determined by ultraviolet matrix-assisted laser desorption/ionization tandem mass spectrometry. Rapid Communications in Mass Spectrometry 24, 841-848 (2010).

16. Jackson, T. R. et al. Boronate affinity saccharide electrophoresis: A novel carbohydrate analysis tool. Electrophoresis 29, 4185-91 (2008).

17. James, T. D. et al., WO2010/041037.

18. Fluckiger, R. & Gallop, P. M. Measurement of nonenzymatic protein glycosylation. Methods Enzymol 106, 77-87 (1984).

19. Gould, B. J. & Hall, P. M. m-Aminophenylboronate affinity ligands distinguish between nonenzymically glycosylated proteins and glycoproteins. Clin Chim Acta 163, 225-30 (1987).

20. Pereira Morais, M. P. et al. Analysis of protein glycation using phenylboronate acrylamide gel electrophoresis. Proteomics 10, 48-58 (2010).

21. Pereira Morais, M. P., Fossey, J. S., James, T. D. & van den Eisen, J. M. Analysis of protein glycation using phenylboronate acrylamide gel electrophoresis. Methods Mol Biot 869, 93-109 (2012).

22. Guerin-Dubourg, A., Catan, A., Bourdon, E. & Rondeau, P. Structural modifications of human albumin in diabetes. Diabetes Metab 38, 171-8 (2012).

23. Wells-Knecht, M. C., Thorpe, S. R., Baynes, J. W., Biochemistry 1995, 34, 15134-15141.

24. Rahbar, S., Cell Biochem Biophys 2007, 48, 147-157.

25. Lindsay, R. M., Smith, W., Lee, W. K., Dominiczak, M. H., Baird, J. D., Clin Chim Acta 1997, 263, 239-247.

26. Rahbar, S., Nadler, J. L., Clin Chim Acta 1999, 287, 123-130.

27. Horbowicz, M., in: Ellis, R. H., Black, M., Murdoch, A. J., Hong, T. D. (Eds.), Basic and applied aspects of seed biology. Proceedings of the Fifth International Workshop on Seeds, Reading, UK 10-15 Sep. 1995, Kluwer Academic Publishers 1997.

28. Lahuta, L. B., R.J., G., Zalewski, K., Hedley, C. L., Acta Physiol Plant 2007, 29, 8.

29. Aon, J. C., Caimi, R. J., Taylor, A. H., Lu, Q., et al., Appl Environ Microbiol 2008, 74, 950-958.

30. Geoghegan, K. F., Dixon, H. B., Rosner, P. J., Hoth, L. R., et al., Anal Biochem 1999, 267, 169-184.

31. Yan, Z., Caldwell, G. W., McDonell, P. A., Jones, W. J., et al., Biochem Biophys Res Commun 1999, 259, 271-282.

32. Yan, Z., Caldwell, G. W., McDonell, P. A., Biochem Biophys Res Commun 1999, 262, 793-800.

33. Loske, C., Gerdemann, A., Schepl, W., Wycislo, M., et al., Eur J Biochem 2000, 267, 4171-4178.

34. Jackson, Anal. Biochem. 1991, 196, 238-244.

35. Seyfried et al, Glycobiology 2005, 15, 303-312.

36. Masuoka and Hazen, Electrophoresis 2006, 27, 365-372.

37. Leonil, J., Molle, D., Fauquant, J., Maubois, J. L., et al., J Dairy Sci 1997, 80, 2270-2281.

38. Morgan, F., Leonil, J., Molle, D., Bouhallab, S., Biochem Biophys Res Commun 1997, 236, 413-417.

39. Howlett, D. R. et al. Cognitive correlates of Abeta deposition in male and female mice bearing amyloid precursor protein and presenilin-1 mutant transgenes. Brain Res 1017, 130-6 (2004).

40. Choudhry, F., Howlett, D. R., Richardson, J. C., Francis, P. T. & Williams, R. J. Pro-oxidant diet enhances beta/gamma secretase-mediated APP processing in APP/PS1 transgenic mice. Neurobiol Aging 33, 960-968 (2012).

41. Elfeky, S. A. et al. Diol appended quenchers for fluorescein boronic acid. Chem Asian J 5, 581-8 (2010).

42. Jacobson, J. et al. Biomarkers of aging in Drosophila. Aging Cell 9, 466-77 (2010).

43. Matsumoto, Y., Sumiya, E., Sugita, T. & Sekimizu, K. An invertebrate hyperglycemic model for the identification of anti-diabetic drugs. PLoS One 6, e18292 (2011).

44. Howlett, D. R. et al. Cognitive correlates of Abeta deposition in male and female mice bearing amyloid precursor protein and presenilin-1 mutant transgenes. Brain Res 1017, 130-6 (2004).

45. Park, K. H., Jang, W., Kim, K. Y., Kim, J. R. & Cho, K. H. Fructated apolipoprotein A-I showed severe structural modification and loss of beneficial functions in lipid-free and lipid-bound state with acceleration of atherosclerosis and senescence. Biochem Biophys Res Commun 392, 295-300 (2010).

46. Jaleel, A. et al. Identification of Amadori-modified plasma proteins in type 2 diabetes and the effect of short-term intensive insulin treatment. Diabetes Care 28, 645-52 (2005).

47. Dolhofer, R. & Wieland, O. H. Increased glycosylation of serum albumin in diabetes mellitus. Diabetes 29, 417-22 (1980).

48. Siegert, K. J. Carbohydrate metabolism during the pupal molt of the tobacco hornworm, Manduca sexta. Archives of Insect Biochemistry and Physiology 28, 63-78 (1995).

49. Layfield, R. et al. Neurofibrillary tangles of Alzheimer's disease brains contain 14-3-3 proteins. Neurosci Lett 209, 57-60 (1996).

50. Choudhry, F., Howlett, D. R., Richardson, J. C., Francis, P. T. & Williams, R. J. Pro-oxidant diet enhances beta/gamma secretase-mediated APP processing in APP/PS1 transgenic mice. Neurobiol Aging 33, 960-968 (2012).

1. A method of determining the presence, level or amount, or identity of one or more glycated species in a complex sample which includes at least five glycated or non-glycated species, the method comprising:
(a) labelling a sample suspected of containing the glycated species with a labelled boronic acid compound, wherein the labelled boronic acid compound is capable of specifically labelling the glycated species present in the sample and substantially does not label glycosylated and/or non-glycosylated species present in the sample;
(b) separating glycated species present in the sample from non-glycated species by electrophoresis; and
(c) detecting the label to determine the presence, level or amount, or identity of the glycated species in the sample.

2. The method of claim 1, wherein the method comprises determining the amount or level of one or more glycated species in the sample.

3. The method of claim 1, wherein the method comprises determining the level or amount and identity of one or more glycated species in the sample.

4. (canceled)

5. The method of claim 1, wherein the electrophoresis is affinity electrophoresis, gel electrophoresis, capillary electrophoresis, dielectrophoresis, isotachophoresis, and/or two-dimensional electrophoresis.

6. (canceled)

7. The method of claim 1, wherein detecting the glycated species comprises transferring the glycated species to a membrane.

8. The method claim 3, wherein the identifying is by electrophoresis, chromatography, direct visualisation protein staining, anti-body probing and/or mass spectrometry.

9-12. (canceled)

13. The method of claim 1, wherein the detecting in step is by direct visualisation of the label using visible or ultraviolet light.

14. (canceled)

15. The method of claim 1, wherein a the step (c) detection protocol is selected from the group consisting of: protein staining, Western blotting, or mass spectrometry.

16-17. (canceled)

18. The method of claim 1, wherein the step of labelling the sample substantially does not affect the electrophoretic migration properties of the glycated species.

19. (canceled)

20. The method of claim 1, wherein the method does not require the use of additional enrichment or purification techniques.

21. The method of claim 1, wherein the sample comprises the glycated species in addition to a corresponding non-glycated species.

22. The method of claim 1, wherein the method is capable of detecting early stage glycation adducts.

23. The method of claim 1, wherein the sample is a fluid sample, such as blood, sera, saliva, cerebrospinal fluid (CSF), tear fluid, hemolymph, or a solid sample, such as a tissue homogenate of skin, brain or other organ, a food sample, or a product of recombinant protein production.

24. The method of claim 1, wherein the sample is a food product, or a product of recombinant protein production.

25. (canceled)

26. The method of claim 1, wherein the method also enables the study of glycated proteins in the context of other non-affected proteins in fluid and solid biosamples.

27. The method of claim 1, wherein the labelled boronic acid compound comprises a fluorescent label.

28. The method of claim 27, wherein the fluorescently labelled boronic acid compound comprises a fluorone dye, a rhodamine dye, an acridine dye, a cyanine dye, an oxazin dye, a phenanthrine dye or a derivative thereof.

29-44. (canceled)

45. The method of claim 1, wherein the glycated species is a polypeptide.

46. The method of claim 45, wherein the glycated polypeptide comprises glycated glucose, mannose, fructose, maltose or galactose sugars.

47. The method of claim 1, further comprising correlating the presence level or amount of one or more of the glycated species as a marker of a disease, condition or biological process.

48. The method according to claim 47, wherein the disease, condition or biological process is selected from cancer, microbial infection, Alzheimer's disease, diabetes, cardiovascular disease and ageing, including diabetes-related ageing.

49. The method of claim 1, wherein the sample is from an animal model of protein glycation.

50-54. (canceled)

55. The method of claim 1, wherein the detecting step further comprises analysis by mass spectrometry.
